(19) United States
(12) Patent Application Publication
(10) Pub. No.: US 2003/0004329 A1
(43) Pub. Date: Jan. 2, 2003

(54) Gene-trap identification of host cell proteins required for hepatitis C virus replication

(76) Inventor: Alexander Alexandrovich Kolykhalov, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/104,398

(22) Filed: Mar. 22, 2002

Related U.S. Application Data
(60) Provisional application No. 60/278,157, filed on Mar. 23, 2001.

(51) Int. Cl. A61K 31/00; C12Q 1/70; C07H 21/04; C12N 15/09; C12N 15/70; C12N 15/74; C12N 15/63; C12N 15/00; A01N 61/00

(52) U.S. Cl. 536/23.72; 435/5; 514/1; 536/24.1; 435/320.1

(57) ABSTRACT

Provided are methods that facilitate the identification of host cell genes required for the replication of hepatitis C virus. Also provided are methods of identifying compounds that inhibit the activity(ies) of products of these genes required for hepatitis C virus replication in host cells, and that therefore inhibit hepatitis C virus replication in such cells. These compounds are useful as hepatitis C virus antiviral pharmaceutical agents to treat or prevent hepatitis C virus infections in humans. Also provided are novel host cell genes identified by these methods; hepatitis C virus replicons comprising both a positive and a negative selectable marker gene; and cell lines comprising said replicons.

This application claims the benefit of priority of U.S. Provisional Patent Application No. 60/278,157, filed Mar. 23, 2001, the entire contents of which are herein incorporated by reference.

### BACKGROUND OF THE INVENTION

1. Field of the Invention

The present invention relates to the field of antiviral therapy, especially the treatment or prevention of hepatitis C virus (HCV) infection in humans. More particularly, the present invention relates to methods for identifying host cell proteins required for HCV replication, and the discovery of molecules exhibiting HCV antiviral activity utilizing such host cell protein targets for anti-HCV pharmaceutical development. Similar methods can be employed to identify host cell proteins required for replication of other viruses important in human and veterinary medicine.

2. Description of Related Art

Significance of Hepatitis C Virus

Identification in 1989 of the Hepatitis C virus as a major cause of non-A non-B hepatitis (Choo et al. (1989) Science 244:359-362) led to rapid advances in diagnostics, and to the realization of the medical problem posed by this virus. Spread primarily by direct contact with human blood, HCV had already infected millions of people through transfusions or unsafe medical practices. Since then, the rate of spread has been significantly reduced in developed countries, but continues fueled mainly by needle-sharing drug users, and by medical practitioners reusing inadequately sterilized needles, thereby inadvertently infecting millions of patients with the pathogen. At least 3 million to 4 million people worldwide are infected each year (Crabb, C. (2001) Science 294:506-507). Once infected, only about 20% of people clear the virus from their blood-stream; the remainder will harbor HCV the rest of their lives. Worldwide, the number of infected people was estimated at 170 million in 1999 (WHO Weekly Epidemiological Record 49:425-427). Although the overall prevalence of HCV chronicity in the United States is 1.8% (about 4 million people total), certain groups have an increased risk. The 30-50 year old age group has a prevalence of approximately 3-4% (Alter et al. (1999) N. Engl. J. Med. 341:556-562). Of those chronic cases, many may never have symptoms, but 10% to 20% eventually develop liver-destroying cirrhosis or cancer. At present, HCV infection is the leading cause of liver transplantation in the United States. The number of deaths related to HCV infection is predicted to triple in the U.S. during the next decade unless efficient treatment of the disease is available (NIH Consensus (1997) Hepatology 26 (Suppl. 1):2S-10S). There is no vaccine to prevent HCV infection, and the only therapy currently available is a combination treatment with a high dose of interferon-α (IFN-α) and the nucleoside analogue ribavirin. However, even with the best available therapy, only less than half of all patients benefit from the treatment and develop a sustained response.

Molecular Biology of HCV

The identification of the HCV viral genome approximately 10 years ago rapidly led to the delineation of the genomic organization and the structural and biochemical characterization of several virus proteins. HCV has been classified as the sole member of a distinct genus called hepacivirus in the family Flaviviridae, which includes the flaviviruses, the animal pathogenic pestiviruses, and the recently cloned GB virus A (GBV-A), GBV-B, and GBV-C/hepatitis G viruses. These viruses have in common an enveloped particle harboring a plus-strand RNA that, in the case of HCV, has a length of ˜9600 nucleotides. The genome carries a single long open reading frame (ORF) encoding a polyprotein that is co- and post-translationally cleaved into 10 distinct products. Little is known about structure and replication of HCV. Studies have been hampered by the lack of a cell culture system and by the usually low titers of virus in patients' serum. HCV nonstructural proteins and viral RNA have been detected in livers of infected patients or experimentally inoculated chimpanzees, confirming that the liver is a site of HCV replication (Blight et al. (1995) Viral Hep. Rev. 1:143-155). Unfortunately, the amounts of viral proteins and RNA in infected tissues are very low, necessitating the use of highly sensitive, but also more error-prone, detection methods. This may in part explain why the reported number of HCV-positive cells detected in infected liver tissue is contradictory, with estimates varying between less than 5% and up to 100%. In addition, there is some evidence that HCV can also replicate in peripheral blood mononuclear cells (PBMCs) (Cribier et al. (1995) J. Gen. Virol. 76:2485-2491).

The majority of studies analyzing HCV genome functions have been carried out using cell-free translation and transient expression in cell cultures employing partial or whole HCV genome RNA. The translation of the ORF is directed via a 5′ non-translated region (NTR), which spans 341 nucleotides immediately prior to the ORF, and functions as an internal ribosome binding site (IRES). The 3′ NTR was only recently discovered (Kolykhalov et al. (1996) J. Virol. 70:3363-3371; Tanaka et al. (1995) Biochem. Biophys. Res. Comm. 215:744-749). It has an unusual molecular structure composed of a variable sequence following the stop codon of the ORF, a poly(U) tract of heterogeneous length, and a highly conserved 98 nucleotide sequence essential for replication in vivo (Yanagi et al. (1999) Proc. Natl. Acad. Sci. USA 96:2291-2295; Kolykhalov et al. (2000) J. Virol. 74:2046-2051). The structural proteins located in the amino-terminal one-third of the ORF, and the nonstructural proteins are in the remainder. Structural proteins are co-translationally cleaved by cellular signal peptidase(s) to generate core protein (C), followed by two membrane glycoproteins, E1 and E2, and by small hydrophobic protein p7. C is a basic protein believed to be the capsid forming protein; E1 and E2 are putative virion envelope glycoproteins; the function of p7 is mostly unknown. These proteins are followed by nonstructural (NS) proteins NS2-NS5B, which function to form RNA-replication complexes. Two proteases are encoded by the HCV genome, both being required for proteolytic processing of the nonstructural part of the polyprotein to generate mature replicase components (Kolykhalov et al. (2000) J. Virol. 74:2046-2051). NS2 and the amino-terminal domain of NS3 constitute the NS2-3 cysteine proteinase, catalysing cis-cleavage at the NS2/3 site. The role of NS2 itself is not clear: replication of the HCV genome RNA can occur without NS2 (Lohmann, et al. (1999) Science 285:110-113). On the other hand, the enzymatic activity of the NS2-3 protease is essential for viral viability in vivo (Kolykhalov et al. (2000) J. Virol. 74:2046-2051); it is possible that the activity is required for generation of the precise N-terminus of the NS3. NS3 is a bifunctional molecule. A serine-type proteinase is responsible for cleavage at the NS3/4A, NS4A/B, NS4B/5A and NS5A/B sites locates in the amino-terminal ˜180 residues. NTPase and helicase activities essential for replication of the HCV genome are located in the carboxy-terminal two thirds of the protein. NS4A is an essential cofactor of the NS3 proteinase, and is required for efficient polyprotein processing. The function of the hydrophobic NS4B is so far unknown. NS5A is a highly phosphorylated protein that has been shown to interact with PKR, providing a plausible mechanism for the modulation of the host response to interferon (Gale et al. (1997) Virology 230:217-227; Gale et al. (1998) Mol. Cel. Biol. 18:5208-5218). However, the precise function of NS5A is still unknown. NS5B was identified as the RNA-dependent RNA polymerase (RdRp) (Behrens et al. (1996) EMBO Journal 15:12-22; Lohmann et al. (1997) J. Virol. 71:8416-8428; Yuan et al. (1997) Biochem. Biophys. Res. Comm. 232:231-235).

Efficient methods for identifying cellular genes that perform specific functions have been described (Hicks et al. (1997) Nat Genet. 16(4):338-44; Hicks et al. (1995) Methods Enzymol. 254:263-75; Durick et al. (1999) Genome Res. 9(11):1019-25; W. C. Skarnes (2000) Methods Enzymol. 328:592-615; Steel et al. (1998) Hippocampus 8(5):444-57; Ishida et al. (1999) Nucleic Acids Res. 27(24):e35; Klinakis et al. (2000) EMBO Rep. 1(5):416-21).

An HCV replicon molecule that replicates in transfected human liver cells has been synthesized based on the sequence provided by Lohmann et al., Science (1999) 285(5424):110-113. This replicon molecule contains a marker (Neo) for positive selection of cells containing the replicon.

The high prevalence of the hepatitis C virus, the insidious course of the disease, and the poor prognosis for long-term persistent infection make this pathogen a serious medical and economic problem. Thus, there exists a pressing need in the art to discover new pharmaceutical agents for the safe and effective prevention and treatment of HCV infection.

### SUMMARY OF THE INVENTION

Accordingly, the present inventor has developed methodology that facilitates the identification of cellular genes and their corresponding proteins involved in HCV replication by use of the aforementioned replicon system. It is currently believed in the art that what is true for replication of the replicon is also true for replication of hepatitis C virus itself. The protein targets so identified can be employed in assays or rational drug design to identify compounds having HCV antiviral inhibitory activity (“anti-HCV activity”).

Therefore, in one aspect, the present invention provides a method of identifying a host cell gene, expression of which is required for HCV replication in the cell. Such cell can be a eukaryotic cell, particularly a mammalian cell, and more particularly a human cell, such as a hepatocyte, a lymphocyte, or a splenocyte. Such cell can be in vitro or in vivo.

In another aspect, the present invention provides a method of identifying a host cell gene, expression of which is required for replication of an HCV replicon in a cell, particularly a mammalian cell, especially a human cell. Such cell can be in vitro or in vivo.

In another aspect, the present invention provides a method of utilizing gene-trap methodology for identifying a host cell gene, expression of which is required for HCV replication in a cell.

In another aspect, the present invention provides a method of utilizing gene-trap methodology for identifying a host cell gene, expression of which is required for replication of an HCV replicon in a cell.

In another aspect, the present invention provides a method of utilizing retroviral gene-trap constructs for identifying a host cell gene, expression of which is required for HCV replication in a cell.

In another aspect, the present invention provides a method of utilizing retroviral gene-trap constructs for identifying a host cell gene, expression of which is required for replication of an HCV replicon in a cell.

In another aspect, the present invention provides a method of utilizing transposon gene-trap constructs for identifying a host cell gene, expression of which is required for HCV replication in a cell.

In another aspect, the present invention provides a method of utilizing transposon gene-trap constructs for identifying a host cell gene, expression of which is required for replication of an HCV replicon in a cell.

In another aspect, the present invention provides a novel host cell gene required for HCV replication that is identified by any of the foregoing methods.

In a further aspect, the present invention provides a hepatitis C virus antiviral compound identified by the methods described herein.

The method of identifying a gene involved in hepatitis C virus or hepatitis C virus replicon replication in a host cell provided herein comprises:

(1) providing a hepatitis C virus replicon molecule containing one or more selectable marker genes for both positive selection and negative selection;

(2) introducing said hepatitis C virus replicon of step (1) into a host cell, wherein said host cell is insensitive to growth in the presence of an agent used for negative selection, thereby creating a cell line comprising said hepatitis C virus replicon;

(3) introducing into said cell line of step (2) a retrovirus construct or a transposon construct comprising a promoterless gene for positive selection, wherein said promoterless gene is different from said selectable marker gene used for positive selection in step (1), and wherein said retrovirus or transposon construct comprises, in operable linkage, a signal required for integration into a host cell genome; an artificial splice acceptor signal; a signal for transcription termination and polyadenylation (Aₙ); two loxP sites wherein, upon integration into host cell genomic DNA, one loxP site is present upstream of said splice acceptor signal, and the other loxP site is present downstream from said Aₙ; and optionally, an origin for bacterial replication and a selectable marker gene for selection in a bacterial cell;

(4) selecting cells in which said retrovirus construct or said transposon construct has integrated into a host cell gene of said cell;

(5) contacting cells of step (4) with said negative selection agent in order to select cells in which said hepatitis C virus replicon of step (1) has either been lost or is not replicating, and said negative selectable marker gene is not expressed or is expressed at a level insufficient to kill cells in the presence of said negative selection agent, thereby marking a gene of said host cell as being involved in HCV replication in said host cell; and

(6) identifying said marked host cell gene.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### BRIEF DESCRIPTION OF THE DRAWINGS

The above and other aspects, features, and advantages of the present invention will be better understood from the following detailed description taken in conjunction with the accompanying drawing, all of which are given by way of illustration only, and are not limitative of the present invention, in which:

FIG. 1. Schematic diagram of suicidal HCV replicons. As an example of a suicidal gene, the thymidine kinase (TK) gene from human HSV-1 is provided. The top two constructs correspond to a bi-cistronic HCV replicon reported by Lohmann et al. (Science (1999) 285(5424):110-113), except the Neo gene is replaced by a fusion of TK-Neo or Neo-TK. The third construct contains a fusion Neo-Ubiquitin-TK, which upon expression in tissue culture provides an unmodified thymidine kinase. In all three top constructs the expression of a suicidal gene is controlled by the HCV IRES. In the fourth provided construct, the expression of a suicidal gene is driven by the more efficient EMCV IRES. The last construct represents a monocistronic HCV replicon. Translation of a single polyprotein is controlled by the HCV IRES. As a result, the last construct contains only HCV-specific sequences controlling both replication and translation of the replicon. Regions of replicons corresponding to IRES structures, as well as 5′ and 3′ NTRs, are shown by thick lines; the thick black line corresponds to HCV-derived sequences; the thick gray line corresponds to EMCV IRES sequences. HCV nonstructural proteins from NS3 to NS5B are shown by the open box. Sequences corresponding to the TK gene are depicted by black boxes; sequences corresponding to Neo and Ubiquitin genes are shown by boxes colored with different shades of gray.

FIG. 2. Schematic diagram illustrating steps of the method to trap host cell genes required for HCV replication. A. Diagram of a suicidal HCV replicon genome used in the method. Other examples are shown in FIG. 1. B. Cell line established after transfection of an HCV replicon into a permissive cell line and selection with G-418. Each cell contains in the cytoplasm replicating HCV genomes (depicted as scaled down version of that shown in A.) Replication of HCV provides expression of the TK gene included in the HCV replicon genome. Therefore, all cells are sensitive to treatment with gancyclovir. C. The cell line from B, above, is infected with retroviral particles defective for retrovirus replication, and selected for provirus integration. The selection is performed in the absence of G-418. Each surviving cell in such a cell library contains a mutation disrupting one of the host cell genes (note that the host cell genes must be actively transcribed since the provirus does not contain a promoter; see text and FIG. 3 for details). Mutations in host genomic DNA resulting from provirus integration are depicted as stars. In the majority of cells, gene expression is not affected by virtue of expression from the second allele of the gene. Such cells will continue to replicate HCV and express TK. When expression of a gene is affected, and expression of the affected gene is essential for HCV replication, the HCV replicon will not replicate, and, as a consequence, such cell will become TK-negative. D. The library of cells from C. is treated with gancyclovir to select the TK-negative cells. Only TK-negative cells are able to survive and establish colonies.

FIG. 3. Schematic diagram of the retroviral trap construct and effects of provirus integration on gene transcription and RNA splicing. A. A host cell gene before provirus integration. This gene contains exons and introns; exons are spliced together during post-transcriptional RNA maturation to generate a mRNA. B. A cellular gene with integrated provirus DNA. The provirus contains an LTR with an inactivated promoter (Δ enh). The expression of the selectable marker gene (Hygr) is dependent on transcription from a host gene promoter. A splice acceptor signal (SA) is positioned immediately before the Hygr gene. To express Hygr, the provirus must integrate into an intron (downstream of a splice donor (SD) signal). The provirus also contains a signal for transcription termination and polyadenylation (Aₙ). Integration of the provirus results in premature termination of transcription (inactivation of the corresponding host gene). Two loxP sites (shown by filled triangles) flank the SA and Aₙ signals, respectively, and are utilized during validation (see text below). A bacterial replication ori sequence and a gene providing resistance in bacteria (Amp) are also included in the provirus sequence (see text below). The provirus also contains a signal for packaging (not shown), but does not contain intact Gag and Pol genes required for replication and spread of the virus.

FIG. 4. Schematic diagram of validation of a trapped gene. A. and B. depict events during trapping (see detailed explanations in FIG. 3, above). Treatment of a selected cell line with a cell-permeable Cre recombinase initiates a recombination event during which sequences between two loxP sites are excised (A. Nagy (2000) Genetics 26:99-109). C. Host cell DNA after treatment with Cre recombinase. The recombination removes the transcription termination signal Aₙ, and results in production of an RNA transcript that, although still containing the remainder of the provirus sequence in the intron, is spliced to generate the original mRNA. The level of protein translated from this mRNA is restored in the treated cells, rendering the cells competent to support HCV replication.

FIG. 5. Scheme for plasmid rescue and identification of a trapped host cell gene sequence. The retrovirus used for trapping is modified to include between the LTR's an ori of plasmid replication in E. coli and the ampicillin resistance gene (Amp^{r}) for plasmid selection. The provirus also contains a unique restriction site, EcoRI. The next EcoRI site is somewhere upstream of the provirus sequence in surrounding host cell genomic DNA. Chromosomal DNA from a selected clone is digested with EcoRI, ligated, and electroporated into E. coli. Only the DNA fragment containing the ori and Amp^{r} can confer resistance of electroporated bacteria to ampicillin. Such bacteria form colonies on ampicillin-containing agar. Plasmid DNA from the colonies is prepared, and the sequence of the trapped intron upstream of the LTR is determined with an LTR-specific primer.

### DETAILED DESCRIPTION OF THE INVENTION

The following detailed description of the invention is provided to aid those skilled in the in practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

All HCV proteins as well as RNA cis-elements can serve as targets for screening of anti-HCV specific therapeutics to intervene in the disease. Such targets can include known enzymatic activities coded in the viral genome (NS3 serine protease, NS2-3 cysteine protease, helicase, RdRp, and others) which could be cloned separately, expressed in significant quantities in bacteria or in mammalian tissue culture systems, purified, and used for in vitro screening for small molecules inhibiting the corresponding enzymatic activity. With the expansion of our understanding of the details of HCV genome RNA function in replication and translation, defined RNA elements derived from the HCV genome are becoming legitimate targets for therapeutic drug development. These include both the whole IRES element, required for HCV polyprotein translation, and individual stem-loop elements which could be screened for inhibitory binding by small molecules. New technologies such as antisense oligonucleotide inhibitors, ribozymes, and post-transcriptional RNA silencing are presently gaining more attention. Small peptides as well as recombinant antibodies targeting viral proteins are also in development as therapeutics.

However, significant challenges in developing effective anti-HCV drugs include the significant diversity of HCV observed in different isolates (reviewed in Bukh et al., (1995) Sem Liver Dis. 15:41-63). Even within a patient, HCV does not exist as a single entity, but rather as a swarm of variants of a dominant sequence, a phenomenon that has been referred to as “quasispecies.” The production of such a large number of variants is primarily due to the high error rate of the viral RdRp, which is in the range of 10⁻⁴ substitutions per nucleotide. This high error rate is reflected by the high mutation rate observed in patients or experimentally inoculated chimpanzees (Major et al. (1999) J. Virol. 73(4):3317-3325). The high sequence variability is coupled with extremely high levels of virion production in infected patients, as high as 1012 particles per day (Neumann et al. (1998) Science 282:103-107). As a result, the emergence of drug resistant variants constitutes a major problem in development of HCV antiviral drugs (see Cohen and Fauci, (1999) Lancet 354(9180):697-698).

A solution for this problem could be targeting not the viral encoded targets, but rather host cell proteins required for viral replication. HCV, like other viruses, is absolutely dependent on the host cell machinery to reproduce itself. Host cell proteins cannot mutate as rapidly as viral proteins due to inherent mechanisms in eucaryotes that maintain and preserve genetic information. Furthermore, not all host cell proteins could be targeted since inhibition of proteins essential for cell function will result in toxicity to the host.

Thus, the present invention provides a system to identify a host cell protein(s) that is not only absolutely required for replication of HCV (or other viruses), but, at the same time, is not essential for host cell viability. This system utilizes the power of a genome-wide screen to identify such protein(s).

Until the present time, the study of HCV replication has been limited to chimpanzee animal models and human patient blood and tissue samples. The lack of a small animal model for HCV infection or of a tissue culture system for HCV replication has severely hampered the identification of the steps involved in HCV replication in cells and, as a result, the development of effective anti-HCV therapeutics. The existence of a selectable HCV replicon permits the performance of cell culture experiments designed to answer questions concerning HCV interaction with host cell genetic and protein synthetic machinery. The present invention provides highly efficient methods for systematic discovery of nonessential host cell genes required for HCV replication in host cells. These methods of gene identification are highly sensitive, and can identify genes whose expression may be very low in host cells, rendering them impossible to identify by classical methods of molecular biology, such as subtractive hybridization of cellular mRNAs, protein-protein or protein-nucleic acid interactions, etc.

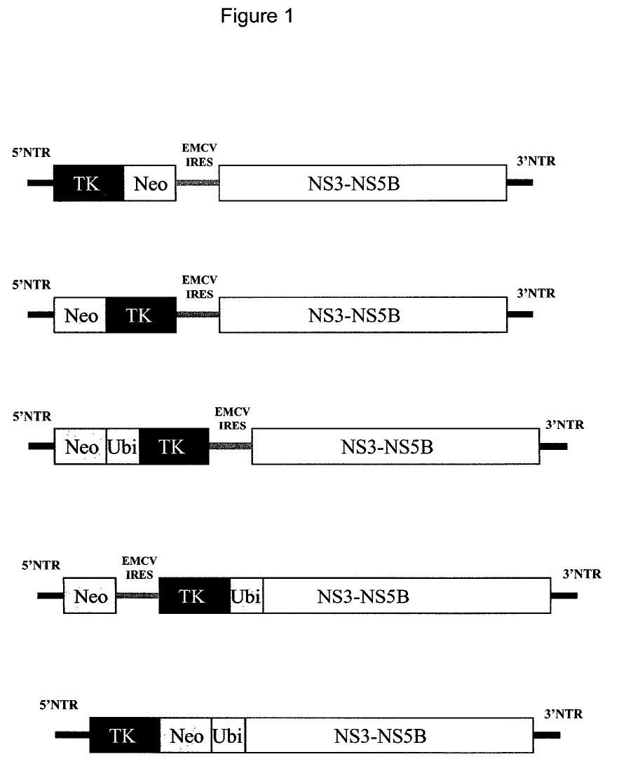

The present invention provides cells harboring a selectable suicidal HCV replicon. The replicon encodes markers both for positive and for negative selection. The cell lines of the present invention are able to maintain the replicon under particular growth conditions, or alternatively, be killed by the same replicon under other controlled growth conditions. For example, conditions under which cells can maintain the replicon include selection with agents such as G-418. Conditions under which cells, containing the replicon, die as a result of replication of the replicon include growth in the presence of selective agents such as aciclovir or ganciclovir. Examples of such replicons are provided in FIG. 1. Application of the present gene-trap hunting method in a cell line harboring an HCV selectable suicidal replicon facilitates identification of host cell genes required for HCV replication. The purpose of identifying such genes is to provide new targets for designing/screening of anti-HCV therapeutics.

As noted above, an HCV replicon molecule capable of replication in transfected human liver cells has been synthesized as reported by Lohmann et al. ((1999) Science 285(5424):110-113). The replicon molecule contains a marker (Neo) for positive selection of cells containing the replicon. This replicon molecule is the basis for construction of a new replicon molecule containing one or more selectable markers for both positive and negative selection in host cells. These markers can be a single selectable marker gene such as TK, which can be used for both positive selection in the presence of HAT, and for negative selection in the presence of ganciclovir or aciclovir. Alternatively, the selectable marker genes can be present as two separate genes (or a fusion of separate genes) wherein each of the separate genes functions to permit selection with a different selection agent, i.e., a positive selection agent and a negative selection agent. In this latter case, the positive selection agent can be, for example, G-418, and the negative selection agent can be, for example, ganciclovir or aciclovir. As used herein, the term “positive selection” refers to the case in which a host cell grown in the presence of a positive selective agent such as G-418 or HAT can survive only when the replicon containing the positive selectable marker gene such as the Neo or TK gene replicates within the cell, and the Neo or TK gene is expressed. As used herein, the term “negative selection” refers to the situation in which a host cell grown in the presence of a negative selective agent such as aciclovir or ganciclovir dies if the replicon containing a suicide gene, such as the herpes simplex virus (HSV) thymidine kinase (TK), replicates within the cell, and the TK gene is expressed. Cell lines that replicate the replicon genome at quite high levels (200-6,000 replicon genomes per cell) can be easily obtained.

Such cell lines can be employed in experiments to identify cellular factors required for HCV replication and to elucidate the mechanisms of HCV replication, with the goal of identifying new targets for anti-HCV intervention, as follows:

1. An HCV replicon molecule containing one or more selectable marker genes for both positive selection and negative selection is constructed (FIG. 1);

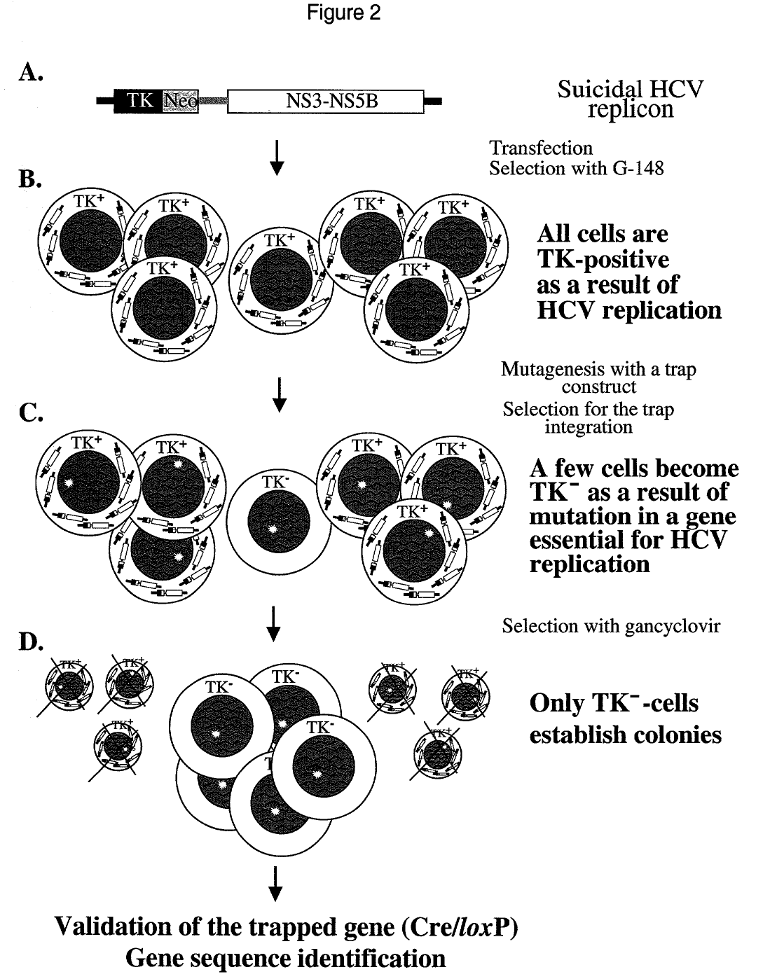

2. The cell line to be used for positive selection of cells containing the replicon should be insensitive to growth in the presence of the agent used for negative selection. For example, the cell line can be TK-negative (insensitive to growth in the presence of aciclovir, ganciclovir, or analogues or derivatives thereof that are activated by the TK). If the cell line is sensitive to the negative selection agent (such as aciclovir ganciclovir, i.e., it is thymidine kinase-positive), then it is necessary to inactivate the gene responsible for activating the negative selection agent (in this case the TK gene) by, for example, targeted mutagenesis. Cells to be used for positive selection, for example TK-negative cells, are transfected with the replicon of Step 1 to create a new cell line that is resistant to the positive selection agent, for example, G-418 (see FIG. 2, B.);

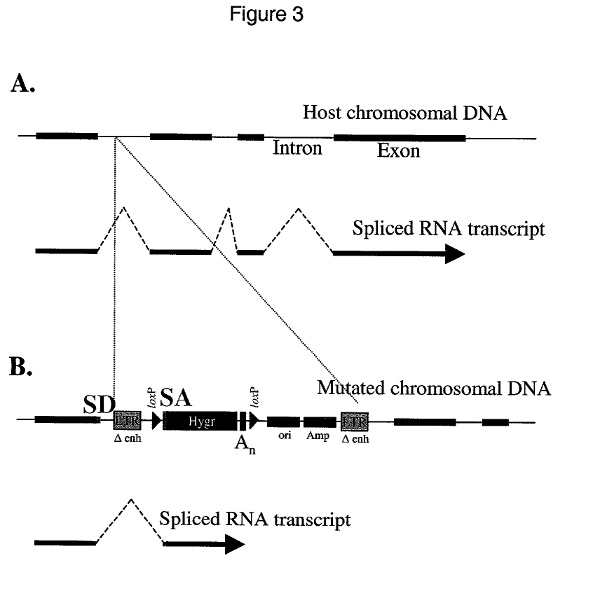

3. A replication-deficient retroviral construct containing a promoterless gene for positive selection (such as the Hyg gene for hygromycin, PAC for puromicin, Bcd for blastocidin, Neo for G-418, or Zeo for zeocin selection) is constructed (FIG. 3) (Friedrich et al. (1991) Genes Devel. 5:1513-1523). It should be noted that the gene used for positive selection in the retroviral construct should be different from the gene used for positive selection of cells containing the HCV replicon. Alternatively, a transposon construct is prepared which similarly contains a promoterless gene for positive selection, as well as an origin for bacterial replication and a selectable marker gene for bacterial selection;

4. The retroviral construct of Step 3 is packaged into retroviral infectious particles using a packaging cell line (A. D. Miller (1992) Curr. Top. Microbiol. Immunol. 158:1-24). The particles can be pseudotyped with VSV-G protein to increase their infectivity for the cells of Step 2 (Abe et al. (1998) J. Virol. 72(8):6356-61; Bartz et al. (1997) Methods 12(4):337-42);

5. The cell line of Step 2 is infected with the retrovirus particles of Step 4, or transfected with the transposon construct of Step 3. Positive selection for cells in which the retrovirus construct or transposon construct has integrated into the host cell genome is performed by incubating cells in the presence of a positive selection agent. Since the expression of the promoterless gene for positive selection in the retroviral construct, or transposon construct, is not driven by its own promoter within the respective construct, expression of this gene is possible only when its ORF is in frame with a host cell gene that is disrupted by integration of the retrovirus or transposon DNA. In addition to all the signals required for retrovirus integration into the host cell genome, the retroviral construct also contains an artificial splice acceptor (SA) site operably linked to the positive selectable marker gene (FIG. 3, B). After viral infection, the provirus integrates randomly into cellular chromosomal DNA. When the integration occurs into an intron, the transcribed RNA will splice such that splice donor (SD) of the preceding exon is joined with the artificial splice acceptor (SA) followed by the selectable marker. If the splicing event does not occur correctly, the cell dies during selection with the antibiotic (hygromycin, in this example). Since the length of introns is about four times as big as that of exons, the trapping of introns is more efficient. The provirus also contains a transcription termination signal (Aₙ). The transcription of RNA is terminated at this signal, resulting in disappearance of mRNA corresponding to the mutated (trapped) gene. As discussed above, only genes nonessential for host cell viability can be trapped this way. If the disrupted host cell gene is required for HCV replication, the replicon in such cell will be lost (FIG. 2, C);

6. The cells of Step 5 are next subjected to negative selection, for example in the presence of aciclovir or ganciclovir, to select cells in which the replicon has either been lost or is not replicating, and the negative selectable marker gene, for example the TK gene, is not expressed or is expressed at a very low level insufficient to kill the cells (FIG. 2, D). If the retrovirus or transposon construct has integrated into a host cell gene that is not required for HCV replication and, consequently, does not result in inhibition of HCV replication, the expression of the negative selectable marker gene (TK) as a result of HCV replication is lethal to the cell in the presence of the negative selection agent (gancyclovir or acyclovir). If the retrovirus or transposon construct has integrated into a host cell gene that is essential for HCV replication, the disruption of such gene will result in inhibition of HCV replication; consequently, the negative selectable marker gene in the replicon is not expressed, and the cell survives in the presence of the negative selection agent. The disrupted gene is “marked” by retrovirus or transposon integration, thereby identifying such host cell gene as being important for HCV replication in the host cell;

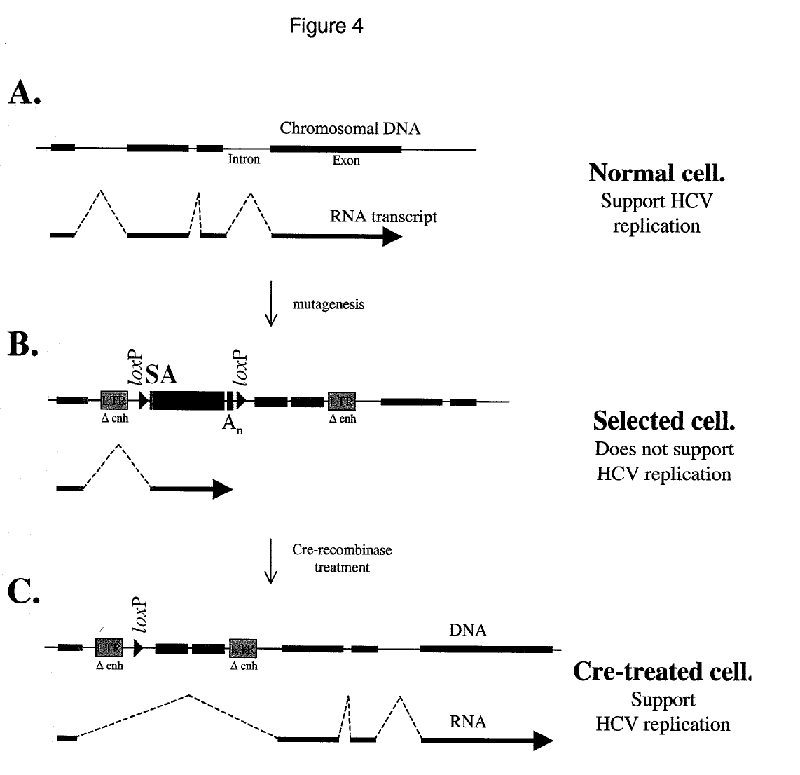

7. Selected clones are validated to demonstrate that the trapped host cell genes therein are required for HCV replication. In addition to the provirus' features described in Step 5, the provirus also contains two loxP sites positioned from one side upstream of the splice acceptor site (SA) and from the other side downstream from the transcription termination signal (Aₙ) (FIG. 3, B, and FIG. 4, B). By providing cell-permeable Cre-recombinase (Jo et al. (2001) Nature Biotechnology 19:929-933), the recombination event is initiated, resulting in deletion from the host cell genome of the artificial SA and Aₙ signals (FIG. 4, C). Consequently, transcription from the gene is restored. Although the resulting RNA transcript still contains in the intron the remainder of the provirus, the transcription is restored (the Aₙ signal has been removed). Since the artificial SA signal has also been removed, the following splicing events generate an mRNA (that is a spliced form of the transcribed RNA) which is identical to the original mRNA sequence (FIG. 4, C). The mRNA is translated, providing to the host cell the protein required for HCV replication. Transfection of HCV replicon RNA into the trapped cell line and into the corresponding cell line “restored” by Cre-loxP recombination reliably discriminates between the “noise” cell line and a true “trapped” cell line: the HCV replicon can replicate only in Cre-treated cells, but not in the “trapped” cell line before treatment (FIG. 4). All other variants are considered to be false-positives. This test is performed in microtiter plate format for higher throughput;

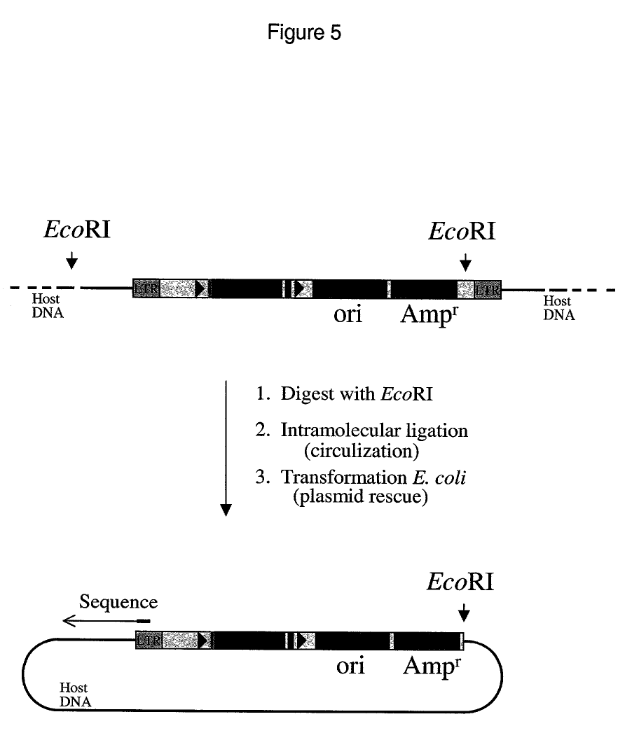

8. Genomic DNA from the validated cell clones of Step 7 is prepared, digested with restriction enzymes, and ligated under optimal conditions for intramolecular circularization (Hicks et al. (1995) Methods in Enzymol. 254:263-75). The retrovirus or transposon construct of Step 3 contains a bacterial replication origin as well as a selectable marker gene, for example an ampicillin resistance gene (FIG. 5). The ligated mixture is electroporated into a transformable host such as E. coli. The ligated DNA containing the ori and Amp gene will survive after transformation into the host upon antibiotic selection. Colonies of bacteria harboring plasmids comprising retrovirus or transposon sequences and the adjacent fragments of the disrupted host cell gene sequences are selected on ampicillin, and the nucleotide sequences of the host cell gene fragments are determined by sequencing of plasmid DNA with LTR-specific primers;

9. Based on the nucleotide sequences obtained in Step 8, the identity of the corresponding host cell genes is determined by, for example, computer genome searching, genome walking techniques, 5′RACE, etc.;

10. Cloning and expression of the identified gene are performed. The functional significance of the gene products for HCV replication is studied by, for example, determining the regulatory function(s) of the gene products, or by determining protein-protein and/or protein-RNA interaction with HCV replication components and/or with cellular pathways important for HCV replication.

11. Screening of small molecules that inhibit the activity of the product of the gene identified in Step 9 and prepared in Step 10 is performed to identify potential HCV antiviral therapeutic compounds. Alternatively, modeling techniques are applied to identify potential binders. Alternatively, cell-permeable therapeutic antibodies are generated to inhibit the activity of the protein product of the gene identified in Step 9. Alternatively, therapeutic antisense oligonucleotides are prepared to inhibit expression of the gene identified in Step 9. Alternatively, therapeutic small interfering RNAs are prepared to inhibit expression of the gene identified in Step 9. Alternatively, small peptides are selected by phage display or by other screening techniques for tight binding to the product of the gene identified in Step 9.

Steps 1-10 lead to the identification of potential targets for inhibition of HCV replication in a cell. Compounds, e.g., biological compounds such as peptides, polypeptides, proteins, peptidomimetics, interfering double stranded RNAs, etc., or small organic molecules, that specifically bind to and/or inhibit or reduce the activity of these target molecules involved in HCV replication or pathogenesis are then identified in Step 11 by in vivo or in vitro binding/assay studies, rational drug design employing crystallized proteins, in vitro or in vivo assays, or by any other methods conventional in the art of pharmaceutical drug discovery. In this way, hepatitis C virus antiviral pharmaceutical agents effective in treating or preventing HCV infections are identified.

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

What is claimed is:

1. A method of identifying a gene involved in hepatitis C virus replication in a host cell, comprising:
(1) providing a hepatitis C virus replicon molecule containing one or more selectable marker genes for both positive selection and negative selection;
(2) introducing said hepatitis C virus replicon of step (1) into a host cell, wherein said host cell is insensitive to growth in the presence of an agent used for negative selection, thereby creating a cell line comprising said hepatitis C virus replicon;
(3) introducing into said cell line of step (2) a retrovirus construct or a transposon construct comprising a promoterless gene for positive selection, wherein said promoterless gene is different from said selectable marker gene used for positive selection in step (1), and wherein said retrovirus or transposon construct comprises, in operable linkage, a signal required for integration into a host cell genome; an artificial splice acceptor signal; a signal for transcription termination and polyadenylation (Aₙ); two loxP sites wherein, upon integration into host cell genomic DNA, one loxP site is present upstream of said splice acceptor signal, and the other loxP site is present downstream from said Aₙ; and optionally, an origin for bacterial replication and a selectable marker gene for selection in a bacterial cell;
(4) selecting cells in which said retrovirus construct or said transposon construct has integrated into a host cell gene of said cell;
(5) contacting cells of step (4) with said negative selection agent in order to select cells in which said hepatitis C virus replicon of step (1) has either been lost or is not replicating, and said negative selectable marker gene is not expressed or is expressed at a level insufficient to kill cells in the presence of said negative selection agent, thereby marking a gene of said host cell as being involved in HCV replication in said host cell; and
(6) identifying said marked host cell gene.

2. A novel host cell gene identified by the method of claim 1.

3. The method of claim 1, further comprising identifying a compound that inhibits the activity of the product of said host cell gene involved in hepatitis C virus replication.

4. A compound identified by the method of claim 3.

5. A hepatitis C virus replicon comprising both a positive and a negative selectable marker gene.

6. A cell line comprising said replicon of claim 5.

7. The cell line of claim 6, wherein said cell line is a hepatocyte cell line.

8. An invention as substantially disclosed herein, including any obvious modification or functional equivalent as would be apparent to one of ordinary skill in the art.
